(19) United States
(12) Patent Application Publication
(10) Pub. No.: US 2019/0053889 A1
(43) Pub. Date: Feb. 21, 2019

(54) ARTIFICIAL IRIS HAVING POUCH

(71) Applicant: XIAMEN EYE CENTER OF XIAMEN UNIVERSITY CO., LTD., Xiamen, Fujian (CN)

(72) Inventors: Haibo Li, Xiamen (CN); Hua Yan, Xiamen (CN); Jinhong Cai, Xiamen (CN); Xiaofeng Li, Xiamen (CN); Duanxiao Wu, Xiamen (CN); Yanming Huang, Xiamen (CN); Donghai Wu, Xiamen (CN); Yan Chen, Xiamen (CN)

(21) Appl. No.: 16/071,908

(22) PCT Filed: Aug. 22, 2017
(86) PCT No.: PCT/CN2017/098464

§ 371 Date: Jul. 23, 2018

(30) Foreign Application Priority Data
Aug. 31, 2016 (CN) 201610790989.X

(51) Int. Cl. A61F 2/14

(52) U.S. Cl. CPC A61F 2/14; A61F 2/1608; A61F 2250/0001; A61F 2220/0025

(57) ABSTRACT

An artificial iris having a pouch includes a top iris, a bottom iris, and more than two fixing rings. The center of the top iris is formed with an inner hole. The outer edges of the top iris and the bottom iris are fixed together to form the pouch. Atop junction of the top iris and the bottom iris is formed with an opening for implanting an intraocular lens. The more than two fixing rings are fixedly mounted on a back surface of the bottom iris. A suture passes through the fixing rings of the artificial iris and is fixed to the scleral wall.

### BACKGROUND OF THE INVENTION

### 1. Field of the Invention

The present invention relates to a medical device, and more particularly, to an artificial iris having a pouch.

### 2. Description of the Prior Art

An artificial iris is often used in an eye without iris or with an injured iris. For injured eyes with good fundus function, it is often necessary to implant an intraocular lens. An intraocular lens with artificial iris is developed on the market, which can simultaneously achieve the role of an artificial iris and an intraocular lens. However, a large corneal incision is required for the intraocular lens to be implanted into the eye, which brings low intraocular pressure during the operation and the risk of explosive choroidal hemorrhage. Furthermore, the postoperative residual astigmatism of the corneal incision causes more corneal neovascularization. The intraocular lens and the artificial iris may be implanted separately. However, secondary implantation requires a scleral flap incision to be fixed through the ciliary sulcus suture, which increases the bleeding and stimulation of the ciliary body.

### SUMMARY OF THE INVENTION

The primary object of the present invention is to provide an artificial iris having a pouch that can be fixed easily and facilitates implantation of an intraocular lens.

In order to achieve the aforesaid object, the present invention adopts the following technical solution:

The present invention relates to an artificial iris having a pouch, comprising a top iris, a bottom iris and more than two fixing rings. A center of the top iris is formed with an inner hole. Outer edges of the top iris and the bottom iris are fixed together to form the pouch. A top junction of the top iris and the bottom iris is formed with an opening for implanting an intraocular lens. The more than two fixing rings are fixedly mounted on a back surface of the bottom iris.

The fixing rings include four fixing rings. The four fixing rings are arranged in pairs parallel to each other. The fixing rings each have a semicircular shape.

The color and texture of a front surface of the top iris are imitated by the color and texture of a healthy iris through a computer acquisition system. The top iris has an outer diameter of 11-13 mm, an inner hole diameter of 3-5 mm, and a thickness of 0.2-0.4 mm.

The fixing rings each have a semicircular shape and a diameter of 0.2 mm.

After adopting the above solution, the back of the artificial iris of the present invention is provided with the fixing rings for fixing the suture that is used to fix the artificial iris. The suture passes through the fixing rings of the artificial iris and is fixed to the scleral wall at a predetermined distance of 1.5 mm spaced apart from the edge of the corneosclera to achieve the purpose of fixation. In addition, the present invention uses acrylic ester as a material to make an ultra-thin artificial iris having a pouch. The artificial iris is folded and implanted in the eye through an incision of 2.8 mm or even a smaller incision. Through the fixing rings on the back of the artificial iris, the polypropylene suture used for fixing the artificial iris is fixed to the sclera of the eyeball. When the intraocular lens is implanted in the second stage, it can be directly implanted in the pouch. There is no need for the intraocular lens to be fixed to the scleral wall of the eyeball again through the polypropylene suture. Besides, because the color of the top iris is consist with the texture and color of the iris of another healthy eye of the patient through a computer acquisition system, ensuring the natural appearance of the eyes of the patient after surgery.

Embodiments of the present invention will now be described, by way of example only, with reference to the accompanying drawings.

### BRIEF DESCRIPTION OF THE DRAWINGS

FIG. 1 is an exploded view of the present invention;

FIG. 2 is a sectional view of the present invention;

FIG. 3 is an exploded view of the present invention mounted with the intraocular lens;

FIG. 4 is a front view of the top iris of the present invention;

FIG. 5 is a rear view of the bottom iris of the present invention;

FIG. 6 is a schematic view of the present invention in a folded state;

FIG. 7 is a front view of the present invention implanted into the eye;

FIG. 8 is a side view of the present invention implanted into the eye; and

FIG. 9 is a schematic view of the present invention implanted with the intraocular lens.

### DETAILED DESCRIPTION OF THE PREFERRED EMBODIMENTS

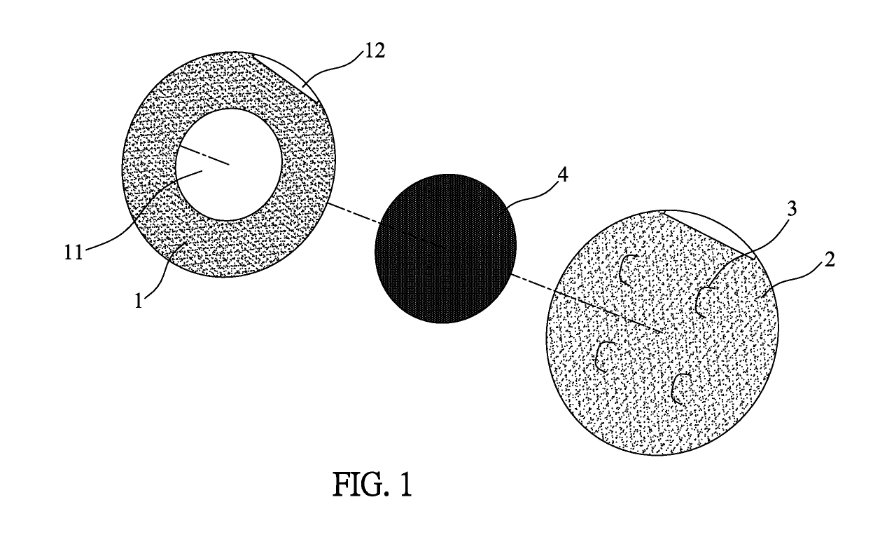

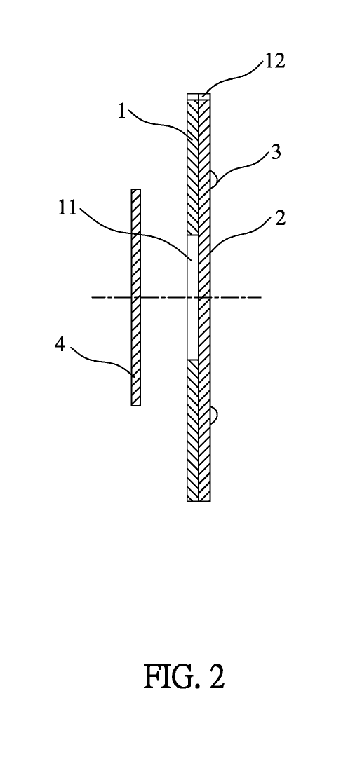

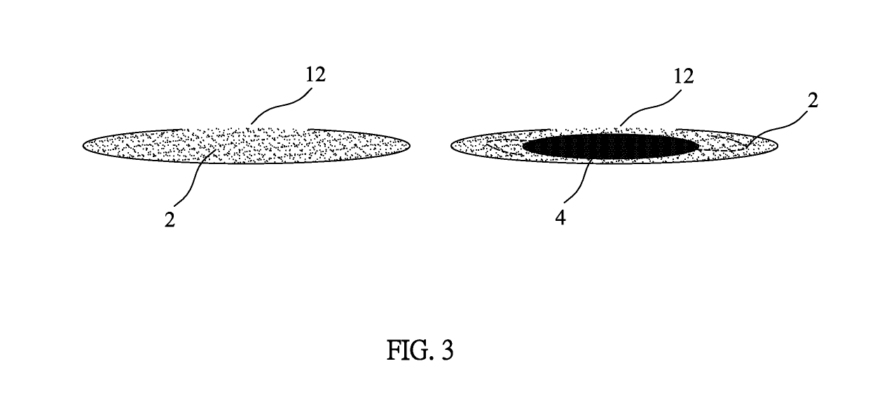

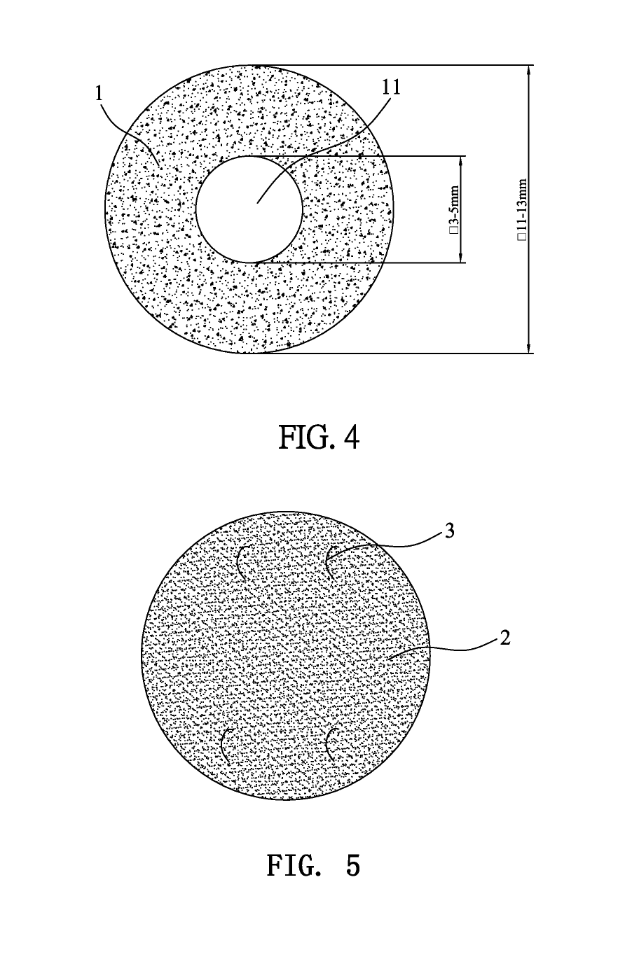

Referring to FIG. 1 to FIG. 5, the present invention relates to an artificial iris having a pouch, comprising a top iris 1, a bottom iris 2 and four fixing rings 3.

The center of the top iris 1 is formed with an inner hole 11. The outer edges of the top iris 1 and the bottom iris 2 are fixed together to form a pouch. Atop junction of the top iris 1 and the bottom iris 2 is formed with an opening 12 for implanting an intraocular lens 4.

The four fixing rings 3 are arranged in pairs parallel to each other. The fixing rings 3 are fixedly mounted on a back surface of the bottom iris 2 respectively, serving as the feet when the artificial iris is fixed by a suture. In this embodiment, the fixing rings 3 each have a semicircular shape and a diameter of 0.2 mm.

The top iris 1 and the bottom iris 2 are made of acrylic ester. The color and texture of a front surface of the top iris 1 are imitated by the color and texture of a healthy iris through a computer acquisition system. The top iris 1 has an outer diameter of 11-13 mm, an inner hole diameter of 3-5 mm, and a thickness of 0.2-0.4 mm.

The surgical procedure of the present invention:

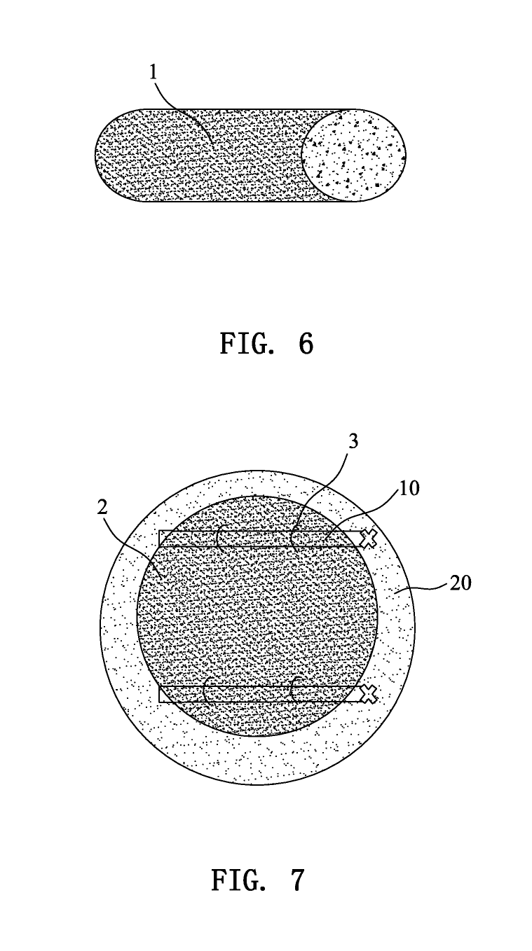

As shown in FIG. 6, at the time of surgery, the iris body 1 is folded and implanted in the eye through an incision of 2.8 mm and then unfolded.

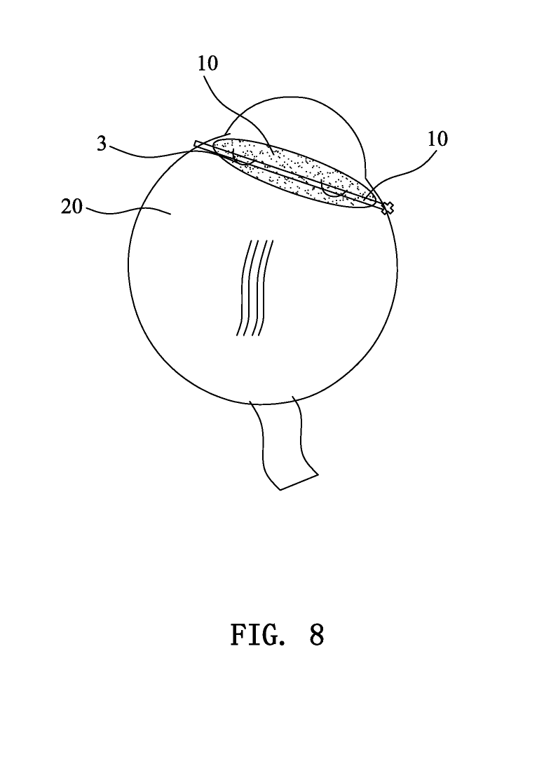

As shown in FIG. 7 and FIG. 8, a suture 10 passes through the four fixing rings 3 of the iris body 1 and is fixed to the scleral wall 20 at a predetermined distance of 1.5 mm spaced apart from the edge of the corneosclera to achieve the purpose of fixation.

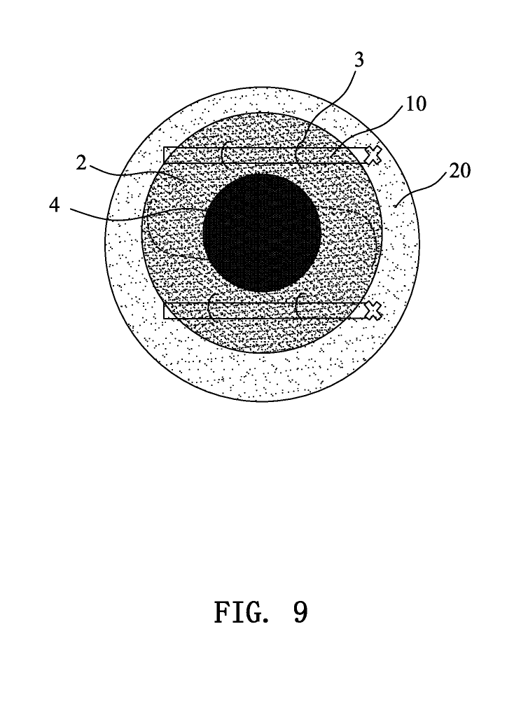

As shown in FIG. 9, the intraocular lens is implanted through the opening 12 between the top iris 1 and the bottom iris 2 without additional fixation.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. An artificial iris having a pouch, comprising a top iris, a bottom iris and more than two fixing rings; a center of the top iris being formed with an inner hole, outer edges of the top iris and the bottom iris being fixed together to form the pouch, a top junction of the top iris and the bottom iris being formed with an opening for implanting an intraocular lens; the more than two fixing rings being fixedly mounted on a back surface of the bottom iris.

2. The artificial iris as claimed in claim 1, wherein the fixing rings include four fixing rings, the four fixing rings are arranged in pairs parallel to each other, and the fixing rings each have a semicircular shape.

3. The artificial iris as claimed in claim 1, wherein the color and texture of a front surface of the top iris are imitated by the color and texture of a healthy iris through a computer acquisition system, and the top iris has an outer diameter of 11-13 mm, an inner hole diameter of 3-5 mm and a thickness of 0.2-0.4 mm.

4. The artificial iris as claimed in claim 1, wherein the fixing rings each have a semicircular shape and a diameter of 0.2 mm.
